SEQUENCE LISTING (20 sequences)

SEQ ID NO: 1 - protein, 5 aa, Homo sapiens
TYGMH

SEQ ID NO: 2 - protein, 17 aa, Homo sapiens
VIWDDGSYKYYGDSVKG

SEQ ID NO: 3 - protein, 16 aa, Homo sapiens
DGITMVRGVMKDYFDY

SEQ ID NO: 4 - protein, 11 aa, Homo sapiens
RASQDISSALV

SEQ ID NO: 5 - protein, 7 aa, Homo sapiens
DASSLES

SEQ ID NO: 6 - protein, 9 aa, Homo sapiens
QQFNSYPLT

SEQ ID NO: 7 - protein, 5 aa, Homo sapiens
SYGIS

SEQ ID NO: 8 - protein, 17 aa, Homo sapiens
WISAYNGYTNYAQKLQG

SEQ ID NO: 9 - protein, 10 aa, Homo sapiens
DLRGTNYFDY

SEQ ID NO: 10 - protein, 11 aa, Homo sapiens
RASQGISNWLA

SEQ ID NO: 11 - protein, 7 aa, Homo sapiens
AASSLLS

SEQ ID NO: 12 - protein, 9 aa, Homo sapiens
QQANSFPIT

SEQ ID NO: 13 - protein, 125 aa, Homo sapiens [sequence omitted]

SEQ ID NO: 14 - protein, 107 aa, Homo sapiens [sequence omitted]

SEQ ID NO: 15 - protein, 119 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Homo sapiens
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Homo sapiens
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0257139 A1
(43) Pub. Date: Aug. 14, 2025

(54) Use of Amivantamab to Treat Colorectal Cancer

(71) Applicant: Janssen Biotech, Inc., Horsham, PA (US)

(72) Inventor: Roland Knoblauch, Philadelphia, PA (US)

(21) Appl. No.: 19/028,911

(22) Filed: Jan. 17, 2025

Related U.S. Application Data
(63) Continuation of application No. 18/077,318, filed on Dec. 8, 2022, now abandoned.
(60) Provisional application No. 63/287,557, filed on Dec. 9, 2021.
(60) Provisional application No. 63/290,765, filed on Dec. 17, 2021.

(51) Int. Cl. C07K 16/28; A61P 35/00

(52) U.S. Cl. CPC C07K 16/2863; A61P 35/00; C07K 2317/31; C07K 2317/565

(57) ABSTRACT

The present invention relates to methods of treating colorectal cancer (CRC), such as metastatic colorectal cancer (mCRC), in a subject in need thereof, comprising administering a therapeutically effective amount of an antibody (e.g., a bispecific antibody) to the subject, wherein the antibody specifically binds epidermal growth factor receptor (EGFR) and hepatocyte growth factor receptor (c-Met).

### CROSS-REFERENCE TO RELATED APPLICATIONS

This application is a continuation of U.S. application Ser. No. 18/077,318, filed on Dec. 8, 2022, which claims the benefit of U.S. Provisional Patent Application No. 63/287,537, filed on Dec. 9, 2021, and U.S. Provisional Patent Application No. 63/290,765, filed on Dec. 17, 2021, the disclosures of each of which are hereby incorporated by reference in their entireties.

### REFERENCE TO SEQUENCE LISTING SUBMITTED ELECTRONICALLY

The sequence listing of the present application is submitted electronically via The United States Patent and Trademark Center Patent Center as an XML formatted sequence listing with a rile name “JB16688USNP1SEQLIST.xml” creation date of Dec. 5, 2022, and a sire of 20 kilobytes (KB). This sequence listing submitted is part of the specification and is herein incorporated by reference in its entirety.

### FIELD OF THE INVENTION

The present invention relates to methods of treating colorectal cancer (CRC), such as metastatic colorectal cancer (mCRC), in a subject in need thereof, comprising administering a therapeutically effective amount of an antibody (e.g., a bispecific antibody) to the subject, wherein the antibody specifically binds epidermal growth factor receptor (EGFR) and hepatocyte growth factor receptor (c-Met).

### BACKGROUND

Colorectal cancer (CRC) is characterized by the unchecked division of abnormal cells in the colon or rectum. CRC is one of the most common malignant neoplasms, ranked 2nd to 4th in terms of incidence in the world, depending on the location, type or gender (Sawicki et al., Cancers (Basel) 13(9):2025 (2021)). CRC is the third most common cause of cancer mortality in the United States (Biller & Schrag, JAMA 325(7):669-85 (2021)). Among people diagnosed with metastatic colorectal cancer (mCRC), fewer than 20% of patients survive beyond 5 years from diagnosis, and the 5-year survival rate for metastatic CRC is 14% (Id.).

### SUMMARY

There is a need for improved therapeutics or combinations of therapeutics to develop more effective treatment of colorectal cancer (CRC), including metastatic CRC (mCRC).

The disclosure generally relates to methods that are useful for treating CRC (e.g., metastatic colorectal cancer (mCRC)).

In one aspect, the disclosure provides a method of treating CRC in a subject in need thereof, comprising administering a therapeutically effective amount of an anti-epidermal growth factor receptor (EGFR)/hepatocyte growth factor receptor (c-Met) antibody to the subject.

In some embodiments, the antibody is a bispecific antibody.

In some embodiments, the antibody (e.g., bispecific antibody) comprises:
a) a first domain that specifically binds EGFR, comprising heavy chain complementarity determining region 1 (HCDR1), HCDR2, HCDR3, light chain complementarity determining region 1 (LCDR1), LCDR2 and LCDR3 amino acid sequences of SEQ ID NOs:1, 2, 3, 4, 5 and 6, respectively; and
b) a second domain that specifically binds c-Met, comprising HCDR1, HCDR2, HCDR3, LCDR1, LCDR2 and LCDR3 amino acid sequences of SEQ ID NOs:7, 8, 9, 10, 11 and 12, respectively.

In certain embodiments, the first domain comprises a heavy chain variable region (VH) of SEQ ID NO:13 and a light chain variable region (VL) of SEQ ID NO:14, and the second domain comprises a VH of SEQ ID NO:15 and a VL of SEQ ID NO:16.

In particular embodiments, the antibody (e.g., bispecific antibody) comprises a first heavy chain (HC1) of SEQ ID NO:17, a first light chain (LC1) of SEQ ID NO:18, a second heavy chain (HC2) of SEQ ID NO:19 and a second light chain (LC2) of SEQ ID NO:20.

In some embodiments, the antibody (e.g., bispecific antibody) is of the IgG1 isotype. In certain embodiments, the antibody (e.g., bispecific antibody) comprises a biantennary glycan structure with a fucose content of about 1% to about 15%. In particular embodiments, the antibody (e.g., bispecific antibody) is Amivantamab.

In some embodiments, the antibody (e.g., bispecific antibody) is administered at a dose of about 700 mg to about 1,400 mg. In certain embodiments, the antibody (e.g., bispecific antibody) is administered once a week or once every two weeks. In particular embodiments, the antibody (e.g., bispecific antibody) is administered once weekly for the first 4 weeks and then every 2 weeks. In some embodiments, the antibody (e.g., bispecific antibody) is administered on a 28-day cycle.

In some embodiments, the antibody (e.g., bispecific antibody) is administered as a monotherapy. In other embodiments, the method further comprises administering one or more chemotherapeutic agents to the subject. In certain embodiments, the one or more chemotherapeutic agents comprise FOLFOX, wherein FOLFOX comprises folinic acid, fluorouracil and oxaliplatin. In certain embodiments, the one or more chemotherapeutic agents comprise FOLFIRI, wherein FOLFIRI comprises folinic acid, fluorouracil and irinotecan.

In some embodiments, the CRC is mCRC. In certain embodiments, the subject has been diagnosed with left-sided mCRC. In other embodiments, the subject has been diagnosed with right-sided mCRC.

In some embodiments, the subject is anti-EGFR therapy naïve. In other embodiments, the subject has received prior anti-EGFR therapy. In certain embodiments, the subject is relapsed or resistant to treatment with one or more prior anti-cancer therapies. In some embodiments, the subject is treatment naïve. In some embodiments, the subject has not received oxaliplatin-based chemotherapy in a metastatic setting.

In some embodiments, the subject is 18 years of age or older. In some embodiments, the subject has been characterized with wild-type KRAS, NRAS and BRAF.

In another aspect, the disclosure provides a method of treating mCRC in a subject, comprising administering a therapeutically effective amount of an isolated bispecific anti-EGFR/c-Met antibody to the subject, wherein the bispecific anti-EGFR/c-Met antibody comprises a first domain that specifically binds EGFR and a second domain that specifically binds c-Met, wherein the first domain comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In another aspect, the disclosure provides a method of treating mCRC in a subject, comprising administering a therapeutically effective amount of an isolated bispecific anti-EGFR/c-Met antibody to the subject, wherein the bispecific anti-EGFR/c-Met antibody comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

In another aspect, the disclosure provides a method of treating mCRC in a subject, comprising administering a therapeutically effective amount of an isolated bispecific anti-EGFR/c-Met antibody to the subject, wherein the bispecific anti-EGFR/c-Met antibody comprises a HC1 of SEQ ID NO: 17, a LC1 of SEQ ID NO: 18, a HC2 of SEQ ID NO: 19 and a LC2 of SEQ ID NO: 20.

In another aspect, the disclosure provides a method of treating mCRC in a subject, comprising administering a therapeutically effective amount of an isolated bispecific anti-EGFR/c-Met antibody to the subject, wherein the bispecific anti-EGFR/c-Met antibody is amivantamab.

### BRIEF DESCRIPTION OF THE DRAWINGS

FIG. 1 shows a study design for Phase 1b/2, open-label study of amivantamab in patients with advanced or metastatic colorectal cancer,

FIG. 2 shows a schematic of the structure of amivantamab, an EGFR and cMet bispecific antibody.

### DETAILED DESCRIPTION

### Definitions

All publications, including but not limited to patents and patent applications, cited in this specification are herein incorporated by reference as though fully set forth.

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising.” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

The term “antibody” or “antibodies” is meant in a broad sense and includes immunoglobulin molecules including monoclonal antibodies including murine, human, humanized and chimeric monoclonal antibodies, full-length antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity.

“Specific binding” or “specifically binds” or “specifically binding” or “binds” refer to an antibody binding to an antigen or an epitope within the antigen with greater affinity than for other antigens. Typically, the antibody binds to the antigen or the epitope within the antigen with an equilibrium dissociation constant (K_{D}) of about 5×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The dissociation constant may be measured using known protocols. Antibodies that bind to the antigen or the epitope within the antigen may, however, have cross-reactivity to other related antigens, for example to the same antigen from other species (homologs), such as human or monkey, for example Macaca fascicularis (cynomolgus, cyno) or Pan troglodytes (chimpanzee, chimp). While a monospecific antibody binds one antigen or one epitope, a bispecific antibody binds two distinct antigens or two distinct epitopes.

“Complementarity determining regions” (CDR) are antibody regions that bind an antigen. CDRs may be defined using various delineations such as Kabat (Wu et al. (1970) J Exp Med 132: 211-50) (Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et a. (2003) Dev Comp Immunol 27: 55-77) and AbM (Martin and Thornton (1996) J Bmol Biol 263: 800-15). The correspondence between the various delineations and variable region numbering are described (see e.g., Lefranc et al. (2003) Dev Comp Immunol 27: 55-77; Honegger and Pluckthun, (2001) J Mol Biol 309:657-70; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“Full-length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Antigen binding fragment” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/NL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Humanized antibodies” refers to antibodies in which the antigen binding sites are derived from non-human species and the variable region frameworks are derived from human immunoglobulin sequences. Humanized antibodies may include intentionally introduced mutations in the framework regions so that the framework may not be an exact copy of expressed human immunoglobulin or germline gene sequences.

“Human antibodies” refers to antibodies having heavy and light chain variable regions in which both the framework and the antigen binding site are derived from sequences of human origin. If the antibody contains a constant region or a portion of the constant region, the constant region is also derived from sequences of human origin. Antibodies in which antigen binding sites are derived from a non-human species are not included in the definition of“human antibody.”

A human antibody comprises heavy or light chain variable regions that are derived from sequences of human origin if the variable regions of the antibody are obtained from a system that uses human germline immunoglobulin or rearranged immunoglobulin genes. Non-limiting example systems include human immunoglobulin gene libraries displayed on phage, and transgenic non-human animals such as mice or rats carrying human immunoglobulin loci. A human antibody typically contains amino acid differences when compared to the human germline or rearranged immunoglobulin sequences due to, for example, naturally occurring somatic mutations, intentional substitutions in the framework or antigen binding site, and substitutions introduced during cloning or VDJ recombination in non-human animals. Typically, a human antibody is at least 80% identical in amino acid sequence to an amino acid sequence encoded by a human germline or rearranged immunoglobulin gene. For example, about: 80%, 81%, 82%, 83%, 84%, 85%, 86%, 87%, 88%, 89%, 90a, 91%, 92%, 93%, 94%, 95%, 96%, 97%, 98%, 99% or 100% identical. In some cases, a human antibody may contain consensus framework sequences derived from human framework sequence analyses (see, e.g., Knappik et al., J. Mol. Biol. 296:57-86 (2000)), or synthetic HCDR3 incorporated into human immune-globulin gene libraries displayed on phage (see, e.g., Shi et al., J. Mol. Biol. 397:385-96(2010) and Int. Pat. Publ. No. WO20090/085462).

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Bispecific anti-EGFR/c-Met antibody” or “bispecific EGFR/c-Met antibody” refers to a bispecific antibody having a first domain that specifically binds EGFR and a second domain that specifically binds c-Met. The domains specifically binding EGFR and c-Met are typically VH/VL pairs, and the bispecific anti-EGFR/c-Met antibody is monovalent in terms of binding to EGFR and c-Met.

“Isolated” refers to a homogenous population of molecules (such as synthetic polynucleotides, polypeptides vectors or viruses) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Low fucose” or “low fucose content” as used in the application refers to antibodies with fucose content of about between 1%-15%.

“Normal fucose” or “normal fucose content” as used herein refers to antibodies with fucose content of about over 50%, typically about over 80% or over 85%.

“Recombinant” refers to DNA, antibodies and other proteins that are prepared, expressed, created or isolated by recombinant means when segments from different sources are joined to produce recombinant DNA, antibodies or proteins.

“Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used to formulate the bispecific anti-EGFR/c-Met antibody. These solutions are sterile and generally free of particulate matter. They may be sterilized by conventional, well-known sterilization techniques (e.g., filtration). For parenteral administration, the carrier may comprise sterile water and other excipients may be added to increase solubility or preservation. Injectable suspensions or solutions may also be prepared utilizing aqueous carriers along with appropriate additives. Suitable vehicles and formulations, inclusive of other human proteins, e.g., human serum albumin, are described, for example, in e.g., Remington: The Science and Practice of Pharmacy, 21^{st} Edition, Troy, D. B. ed., Lippincott Williams and Wilkins, Philadelphia, PA 2006, Part 5, Pharmaceutical Manufacturing pp 691-1092, See especially pp. 958-989.

“Dosage” refers to the information of the amount of the therapeutic or the drug to be taken by the subject and the frequency of the number of times the therapeutic is to be taken by the subject. “Dose” refers to the amount or quantity of the therapeutic or the drug to be taken each time.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Co-administration,” “administration with,” “administration in combination with,” “in combination with” or the like, encompass administration of the selected therapeutics or drugs to a single patient, and are intended to include treatment regimens in which the therapeutics or drugs are administered by the same or different route of administration or at the same or different time.

“Fixed combination” refers to a single pharmaceutical composition comprising two or more compounds.

“Non-fixed combination” refers to separate pharmaceutical compositions, wherein each comprises one or more compounds. The one or more compounds or unit dosage forms can be administered as separate entities either simultaneously, concurrently or sequentially with no specific intervening time limits, wherein such administration provides effective levels of the two compounds in the body of the subject.

“PD-(L)1 axis inhibitor” refers to a molecule that inhibits PD-1 downstream signaling. PD-(L)1 axis inhibitor may be a molecule that binds PD-1, PD-L1 or PD-L2.

“Antagonist” or “inhibitor” refers to a molecule that, when bound to a cellular protein, suppresses at least one reaction or activity that is induced by a natural ligand of the protein. A molecule is an antagonist when the at least one reaction or activity is suppressed by at least about 20%, 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist.

“Treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Prevent”, “preventing”, “prevention”, or “prophylaxis” of a disease or disorder means preventing that a disorder occurs in subject.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” are used interchangeably herein.

“Cancer” refers to an abnormal growth of cells which tend to proliferate in an uncontrolled way and, in some cases, to metastasize (spread) to other areas of a patient's body.

“EGFR or c-Met expressing cancer” refers to cancer that has detectable expression of EGFR or c-Met or has EGFR or c-Met mutation or amplification. EGFR or c-Met expression, amplification and mutation status can be detected using know methods, such as sequencing, next generation sequencing, fluorescent in situ hybridization, immunohistochemistry, flow cytometry or western blotting.

“Epidermal growth factor receptor” or “EGFR” refers to the human EGFR (also known as HER1 or ErbB1 (Ullrich et al., Nature 309:418-425, 1984) having the amino acid sequence shown in GenBank accession number NP_005219, as well as naturally-occurring variants thereof.

“Hepatocyte growth factor receptor” or “c-Met” as used herein refers to the human c-Met having the amino acid sequence shown in GenBank Accession No: NP_001120972 and natural variants thereof.

“Newly diagnosed” refers to a subject who has been diagnosed with EGFR or c-Met expressing cancer but has not yet received treatment for CRC (e.g., mCRC).

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Diagnosing” or “diagnosis” refers to methods to determine if a subject is suffering from a given disease or condition or may develop a given disease or condition in the future or is likely to respond to treatment for a prior diagnosed disease or condition, i.e., stratifying a patient population on likelihood to respond to treatment. Diagnosis is typically performed by a physician based on the general guidelines for the disease to be diagnosed or other criteria that indicate a subject is likely to respond to a particular treatment.

“Biological sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium, lavage fluids and the like, tissue biopsies, tumor tissue biopsies, tumor tissue samples, fine needle aspirations, surgically resected tissue, organ cultures or cell cultures.

### Methods of the Disclosure

In one aspect, the disclosure provides a method of treating CRC in a subject in need thereof, comprising administering a therapeutically effective amount of an anti-epidermal growth factor receptor (EGFR)/hepatocyte growth factor receptor (c-Met) antibody to the subject.

### Anti-EGFR/c-Met Antibodies

In some embodiments, the anti-EGFR/c-Met antibody is a bispecific antibody. In certain embodiments, the antibody is an isolated antibody. In particular embodiments, the antibody is an isolated bispecific antibody.

In some embodiments, the antibody (e.g., bispecific antibody) comprises a first domain that specifically binds EGFR and a second domain that specifically binds c-Met.

### EGFR Binding Arm

In some embodiments, the first domain that specifically binds EGFR comprises:
a) heavy chain complementarity determining region 1 (HCDR1), HCDR2, HCDR3 amino acid sequences of SEQ ID NOs:1, 2 and 3, respectively; and/or
b) light chain complementarity determining region 1 (LCDR1), LCDR2 and LCDR3 amino acid sequences of SEQ ID NOs:4, 5 and 6, respectively.

In certain embodiments, the first domain that specifically binds EGFR comprises:
a) HCDR1, HCDR2, HCDR3 amino acid sequences of SEQ ID NOs:1, 2 and 3, respectively; and
b) LCDR1, LCDR2 and LCDR3 amino acid sequences of SEQ ID NOs:4, 5 and 6, respectively.

HCDR1:
(SEQ ID NO: 1)
TYGMH
HCDR2:
(SEQ ID NO: 2)
VIWDDGSYKYYGDSVKG
HCDR3:
(SEQ ID NO: 3)
DGITMVRGVMKDYFDY
HCDR1:
(SEQ ID NO: 4)
RASQDISSALV
HCDR2:
(SEQ ID NO: 5)
DASSLES
HCDR3:
(SEQ ID NO: 6)
QQFNSYPLT

In some embodiments, the first domain comprises a heavy chain variable region (VH) amino acid sequence that is at least 90% identical to SEQ ID NO:13, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:13. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the first domain comprises a VH of SEQ ID NO:13.

In certain embodiments, the first domain comprises a light chain variable region (VL) amino acid sequence that is at least 90% identical to SEQ ID NO:14, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:14. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the first domain comprises a VL of SEQ ID NO:14.

As used herein, the term “identical” or “has sequence identity,” refers to the extent to which two amino acid sequences have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same amino acid upon alignment of the reference and test sequences to achieve a maximal level of identity. As an example, two sequences are considered to have 70% sequence identity when, upon alignment to achieve a maximal level of identity, the test sequence has the same amino acid residue at 70% of the same positions over the entire length of the reference sequence.

In some embodiments, the first domain comprises:
a) a VH amino acid sequence that is at least 90% identical to SEQ ID NO:13; and/or
b) a VL amino acid sequence that is at least 90% identical to SEQ ID NO:14.

In some embodiments, the first domain comprises:
a) a VH amino acid sequence that is at least 90% identical to SEQ ID NO:13; and
b) a VL amino acid sequence that is at least 90% identical to SEQ ID NO:14.

In some embodiments, the first domain comprises:
a) a VH of SEQ ID NO:13; and/or
b) a VL of SEQ ID NO:14.

In particular embodiments, the first domain comprises:
a) a VH of SEQ ID NO:13; and
b) a VL of SEQ ID NO:14.

VH:
(SEQ ID NO: 13)
QVQLVESGGGVVQPGRSLRLSCAASGFTFSTYGMHWVRQAPGKGLE
WVAVIWDDGSYKYYGDSVKGRFTISRDNSKNTLYLQMNSLRAEDTAVYY
CARDGITMVRGVMKDYFDYWGQGTLVTVSS
VL:
(SEQ ID NO: 14)
AIQLTQSPSSLSASVGDRVTITCRASQDISSALVWYQQKPGKAPKLLIY
DASSLESGVPSRFSGSESGTDFTLTISSLQPEDFATYYCQQFNSYPLTF
GGGTKVEIK

In some embodiments, the first domain comprises a first heavy chain (HC1) amino acid sequence that is at least 80% identical to SEQ ID NO:17, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:17. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the first domain comprises a HC1 amino acid sequence of SEQ ID NO:17.

In some embodiments, the first domain comprises a first light chain (LC1) amino acid sequence that is at least 80% identical to SEQ ID NO:18, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:18. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the first domain comprises a LC1 amino acid sequence of SEQ ID NO:18.

In some embodiments, the first domain comprises:
a) a HC1 amino acid sequence that is at least 80% identical to SEQ ID NO:17; and/or
b) a LC1 amino acid sequence that is at least 80% identical to SEQ ID NO:18.

In some embodiments, the first domain comprises:
a) a HC1 amino acid sequence that is at least 80% identical to SEQ ID NO:17; and
b) a LC1 amino acid sequence that is at least 80% identical to SEQ ID NO:18.

In some embodiments, the first domain comprises:
a) a HC1 of SEQ ID NO:17; and/or
b) a LC1 of SEQ ID NO:18.

In some embodiments, the first domain comprises:
a) a HC1 of SEQ ID NO:17; and
b) a LC1 of SEQ ID NO:18.

HC1:
(SEQ ID NO: 17)
QVQLVESGGGVVQPGRSLRLSCAASGFTFSTYGMHWVRQAPGKGL
EWVAVIWDDGSYKYYGDSVKGRFTISRDNSKNTLYLQMNSLRAEDTAVY
YCARDGITMVRGVMKDYFDYWGQGTLVTVSSASTKGPSVFPLAPSSKST
SGGTAALGCLVKDYFPEPVTVSWNSGALTSGVHTFPAVLOSSGLYSLSS
VVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVE
WESNGQPENNYKTTPPVLDSDGSFLLYSKLTVDKSRWQQGNVFSCSVMH
EALHNHYTQKSLSLSPQK
LC1:
(SEQ ID NO: 18)
AIQLTQSPSSLSASVGDRVTITCRASQDISSALVWYQQKPGKAPKLLI
YDASSLESGVPSRFSGSESGTDFTLTISSLQPEDFATYYCQQFNSYPLT
FGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

c-Met Binding Arm

In certain embodiments, the second domain that specifically binds c-Met comprises:
a) HCDR1, HCDR2, HCDR3 amino acid sequences of SEQ ID NOs:7, 8 and 9, respectively; and/or
b) LCDR1, LCDR2 and LCDR3 amino acid sequences of SEQ ID NOs:10, 11 and 12, respectively.

In certain embodiments, the second domain that specifically binds c-Met comprises:
a) HCDR1, HCDR2, HCDR3 amino acid sequences of SEQ ID NOs:7, 8 and 9, respectively; and
b) LCDR1, LCDR2 and LCDR3 amino acid sequences of SEQ ID NOs:10, 11 and 12, respectively.

HCDR1:
(SEQ ID NO: 7)
SYGIS
HCDR2:
(SEQ ID NO: 8)
WISAYNGYTNYAQKLQG
HCDR3:
(SEQ ID NO: 9)
DLRGTNYFDY
HCDR1:
(SEQ ID NO: 10)
RASQGISNWLA
HCDR2:
(SEQ ID NO: 11)
AASSLLS
HCDR3:
(SEQ ID NO: 12)
QQANSFPIT

In some embodiments, the second domain comprises a VH amino acid sequence that is at least 90% identical to SEQ ID NO:15, e.g., about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:15. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the second domain comprises a VH of SEQ ID NO:15

In certain embodiments, the second domain comprises a VL amino acid sequence that is at least 90% identical to SEQ ID NO:16, e.g., about: 91%, 92%, 93%, 94%, 95%, 90%, 97%, 98%, 99%, 99.1%, 99.2%, 99.3%, 99.4%, 99.5%, 99.6%, 99.7%, 99.8%, or 99.9% identical to SEQ ID NO:16. In some embodiments, the sequence identity is about: 90-99.9%, 90-99.8%, 92-99.8%, 92-99.6%, 94-99.6%, 94-99.5%, 95-99.5%, 95-99.4%, 96-99.4%, 96-99.2%, 97-99.2% or 97-99%. In particular embodiments, the second domain comprises a VL of SEQ ID NO:16.

In some embodiments, the second domain comprises:
a) a VH amino acid sequence that is at least 90% identical to SEQ ID NO: 15; and/or
b) a VL amino acid sequence that is at least 90% identical to SEQ ID NO:16.

In some embodiments, the second domain comprises:
a) a VH amino acid sequence that is at least 90% identical to SEQ ID NO:15; and
b) a VL amino acid sequence that is at least 90% identical to SEQ ID NO:16.

In some embodiments, the second domain comprises:
a) a VH of SEQ ID NO:15; and/or
b) a VL of SEQ ID NO: 16.

In particular embodiments, the second domain comprises:
a) a VH of SEQ ID NO:15; and
b) a VL of SEQ ID NO:16,

VH:
(SEQ ID NO: 15)
QVQLVQSGAEVKKPGASVKVSCETSGYTFTSYGISWVRQAPGHGLE
WMGWISAYNGYTNYAQKLQGRVTMTTDTSTSTAYMELRSLRSDDTAVYY
CARDLRGTNYFDYWGQGTLVTVSS
VL:
(SEQ ID NO: 16)
DIQMTQSPSSVSASVGDRVTITCRASQGISNWLAWFQHKPGKAPKLLI
YAASSLLSGVPSRFSGSGSGTDFTLTISSLQPEDFATYYCQQANSFPIT
FGQGTRLEIK

In some embodiments, the second domain comprises a second heavy chain (HC2) amino acid sequence that is at least 80% identical to SEQ ID NO:19, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:19. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the second domain comprises a HC2 amino acid sequence of SEQ ID NO:19.

In some embodiments, the second domain comprises a second light chain (LC2) amino acid sequence that is at least 80% identical to SEQ ID NO:20, e.g., about: 81%, 82%, 83%, 84%, 85%, 86%, 87%, 88%, 89%, 90%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:20. In certain embodiments, the sequence identity is about: 80-99.9%, 80-99.8%, 85-99.8%, 85-99.6%, 90-99.6%, 90-99.5%, 95-99.5%, 95-99.4%, 96-99.4%, 96-99.2%, 97-99.2% or 97-99%. In particular embodiments, the second domain comprises a LC2 amino acid sequence of SEQ ID NO:20.

In some embodiments, the second domain comprises:
a) a HC2 amino acid sequence that is at least 80% identical to SEQ ID NO:19; and/or
b) a LC2 amino acid sequence that is at least 800/a identical to SEQ ID NO:20.

In some embodiments, the second domain comprises:
a) a HC2 amino acid sequence that is at least 80% identical to SEQ ID NO:19; and
b) a LC2 amino acid sequence that is at least 80% identical to SEQ ID NO:20.

In some embodiments, the second domain comprises:
a) a HC2 of SEQ ID NO:19; and/or
b) a LC2 of SEQ ID NO:20.

In some embodiments, the second domain comprises:
a) a HC2 of SEQ ID NO:19; and
b) a LC2 of SEQ ID NO:20.

HC2:
(SEQ ID NO: 19)
QVQLVQSGAEVKKPGASVKVSCETSGYTFTSYGISWVRQAPGHGLE
WMGWISAYNGYTNYAQKLQGRVTMTTDTSTSTAYMELRSLRSDDTAVYY
CARDLRGTNYFDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQP
ENNYKTTPPVLDSDGSFFLYSRLTVDKSRWQQGNVFSCSVMHEALANHY
TQKSLSLSPGK
LC2:
(SEQ ID NO: 20)
DIQMTQSPSSVSASVGDRVTITCRASQGISNWLAWFQHKPGKAPKLL
IYAASSLLSGVPSFSGSGSGTDFTLTISSLQPEDFATYYCQQANSFPIT
FGQGTRLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

In some embodiments, the antibody (e.g., bispecific antibody) comprises:
a) a first domain that specifically binds EGFR, comprising HCDR1, HCDR2, HCDR3, LCDR1, LCDR2 and LCDR3 amino acid sequences of SEQ ID NOs:1, 2, 3, 4, 5 and 6, respectively; and/or
b) a second domain that specifically binds c-Met, comprising HCDR1, HCDR2, HCDR3, LCDR1, LCDR2 and LCDR3 amino acid sequences of SEQ ID NOs:7, 8, 9, 10, 11 and 12, respectively.

In certain embodiments, the antibody (e.g., bispecific antibody) comprises:
a) a first domain that specifically binds EGFR, comprising HCDR1, HCDR2, HCDR3, LCDR1, LCDR2 and LCDR3 amino acid sequences of SEQ ID NOs:1, 2, 3, 4, 5 and 6, respectively; and
b) a second domain that specifically binds c-Met, comprising HCDR1, HCDR2, HCDR3, LCDR1, LCDR2 and LCDR3 amino acid sequences of SEQ ID NOs:7, 8, 9, 10, 11 and 12, respectively.

In some embodiments, the antibody (e.g., bispecific antibody) comprises:
a) a first domain comprising a VH amino acid sequence that is at least 90% identical to SEQ ID NO:13;
b) a first domain comprising a VL amino acid sequence that is at least 90% identical to SEQ ID NO:14;
c) a second domain comprising a VH amino acid sequence that is at least 90% identical to SEQ ID NO:15; and/or
d) a second domain comprising a VL amino acid sequence that is at least 90% identical to SEQ ID NO:16.

In certain embodiments, the antibody (e.g., bispecific antibody) comprises:
a) a first domain comprising a VH amino acid sequence that is at least 90% identical to SEQ ID NO:13;
b) a first domain comprising a VL amino acid sequence that is at least 90% identical to SEQ ID NO:14;
c) a second domain comprising a VH amino acid sequence that is at least 90% identical to SEQ ID NO:15; and
d) a second domain comprising a VL amino acid sequence that is at least 90% identical to SEQ ID NO:16.

In some embodiments, the antibody (e.g., bispecific antibody) comprises:
a) a first domain comprising a VH of SEQ ID NO:13;
b) a first domain comprising a VL of SEQ ID NO:14;
c) a second domain comprising a VH of SEQ ID NO:15; and/or
d) a second domain comprising a VL of SEQ ID NO:16.

In some embodiments, the antibody (e.g., bispecific antibody) comprises:
a) a first domain comprising a VH of SEQ ID NO:13;
b) a first domain comprising a VL of SEQ ID NO:14;
c) a second domain comprising a VH of SEQ ID NO:15; and
d) a second domain comprising a VL of SEQ ID NO:16.

In certain embodiments, the antibody (e.g., bispecific antibody) comprises:
a) a HC1 amino acid sequence that is at least 80% identical to SEQ ID NO:17;
b) a LC1 amino acid sequence that is at least 80% identical to SEQ ID NO:18;
c) a HC2 amino acid sequence that is at least 80% identical to SEQ ID NO:19; and/or
d) a LC2 amino acid sequence that is at least 80% identical to SEQ ID NO:20.

In certain embodiments, the antibody (e.g., bispecific antibody) comprises:
a) a HC1 amino acid sequence that is at least 80% identical to SEQ ID NO:17;
b) a LC1 amino acid sequence that is at least 80% identical to SEQ ID NO:18;
c) a HC2 amino acid sequence that is at least 80% identical to SEQ ID NO:19; and
d) a LC2 amino acid sequence that is at least 80/identical to SEQ ID NO:20.

In certain embodiments, the antibody (e.g., bispecific antibody) comprises:
a) a HC1 of SEQ ID NO:17;
b) a LC1 of SEQ ID NO:18;
c) a HC2 of SEQ ID NO:19; and/or
d) a LC2 of SEQ ID NO:20.

In particular embodiments, the antibody (e.g., bispecific antibody) comprises:
a) a 1C1 of SEQ ID NO:17;
b) a LC1 of SEQ ID NO:18;
c) a HC2 of SEQ ID NO:19; and
d) a LC2 of SEQ ID NO:20.

In some embodiments, the antibody (e.g., bispecific antibody) is of the IgG isotype. In certain embodiments, the antibody (e.g., bispecific antibody) is of the IgG1 isotype. Some variation exists within the IgG1 constant domain (e.g., well-known allotypes), for example, with variation at positions 214, 356, 358, 422, 431, 435 and/or 436 (residue numbering according to the EU numbering)(see e.g., IMGT Web resources; IMGT Repertoire (IG and TR); Proteins and alleles; allotypes). The bispecific anti-EGFR/c-Met antibody may be of any IgG1 allotype, such as Glm17, Glm3, Glm1, Glm2, Glm27 or Glm28.

In some embodiments, the antibody is a human antibody.

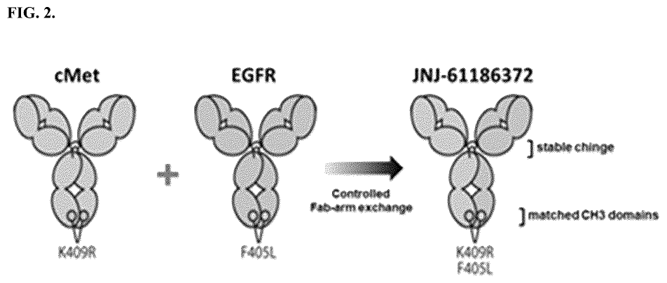

In particular embodiments, the antibody is amivantamab. Amivantamab or JNJ-61186372 (JNJ-372) is an IgG1 anti-EGFR/c-Met bispecific antibody described in U.S. Pat. No. 9,593,164. A schematic of the structure of amivantamab is shown in FIG. 2. The disclosure is based, at least in part, on the finding that amivantamab is effective in treating CRC such as mCRC.

Other anti-EGFR/c-Met antibodies (e.g., bispecific antibodies) may also be used in the methods of the disclosure, for example, by combining publicly available EGFR binding VH/VL domains and c-Met binding VH/VL domains.

In some embodiments, the antibody (e.g., bispecific antibody) comprises a biantennary glycan structure with a fucose content of between about 1% to about 15%.

Antibodies with reduced fucose content can be made using different methods reported to lead to the successful expression of relatively high defucosylated antibodies bearing the biantennary complex-type of Fc oligosaccharides such as control of culture osmolality (Konno et al., Cytotechnology 64(:249-65, 2012), application of a variant CHO line Lec3 as the host cell line (Shields et al., J Biol Chen 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al, J Biol Chem 278:3466-3473, 2003), introduction of small interfering RNA specifically against the α1,6-fucosyltransferase (FUT8) gene (Mori et al., Biotechnol Bioeng 88:901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Zhou ee al., Biotechnol Bioeng 99:652-65, 2008). In general, lowering fucose content in the glycan of the antibodies potentiates antibody-mediated cellular cytotoxicity (ADCC).

### Generating Anti-EGFR/c-Met Antibodies

Anti-EGFR/c-Met antibodies used in the methods of the disclosure may be generated, for example, using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e., an epitope on EGFR and an epitope on c-Met. For example, the bispecific antibodies of the invention may be generated using the technology described in Int. Pat. Publ. No. WO2011/131746. Mutations F405L in one heavy chain and K409R in the other heavy chain may be used in case of IgG1 antibodies. For IgG2 antibodies, a wild-type IgG2 and a IgG2 antibody with F405L and R409K substitutions may be used. For IgG4 antibodies, a wild-type IgG4 and a IgG4 antibody with F405L and R409K substitutions may be used. To generate bispecific antibodies, the first monospecific bivalent antibody and the second monospecific bivalent antibody are engineered to have the aforementioned mutation in the Fc region, and the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Bispecific anti-EGFR/c-Met antibodies used in the methods of the disclosure may also be generated using designs such as the Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), and the Biclonic (Merus).

In the “knob-in-hole” strategy (see, e.g., intl. Publ. No. WO 2006/028936) select amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S L368A_Y407V.

CrossMAb technology, in addition to utilizing the “knob-in-hole” strategy to promote Fab arm exchange utilizes CH1/CL domain swaps in one half arm to ensure correct light chain pairing of the resulting bispecific antibody (see e.g., U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by the following substitutions (expressed as modified positions in the first CH3 domain of the first heavy chain/modified position in the second CH3 domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Patent Publ. No. US2012/0149876 or U.S. Patent Publ. No. US2013/0195849.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

Mutations are typically made at the DNA level to a molecule such as the constant domain of the antibody using standard methods.

### Administration

The anti-EGFR/c-Met antibody (e.g., bispecific antibody) and/or additional therapeutic (e.g., chemotherapeutic) agent may be administered in a pharmaceutical composition or compositions. In some embodiments, the pharmaceutical composition further comprises a pharmaceutically acceptable carrier.

The mode of administration may be any suitable route that delivers the antibody (e.g., bispecific antibody) to the subject in need thereof, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by, for example intratumoral, intracolic, intraabdominal, intragastric, intracavitary, intrapelvic, intraperitoneal, intrarectal, intrathoracic, intravascular, intralesional, rectal, buccal, sublingual, intranasal, or transdermal delivery.

In some embodiments, the pharmaceutical composition comprising the anti-EGFR/c-Met antibody (e.g., bispecific antibody) is administered via an intravenous infusion. In some embodiments, the additional therapeutic (e.g., chemotherapeutic) agent is administered via an intravenous infusion.

In some embodiments, the pharmaceutical composition comprising the anti-EGFR/c-Met antibody (e.g., bispecific antibody) is administered via a subcutaneous injection.

In some embodiments, the antibody (e.g., bispecific antibody) is administered at a dose of about 140 mg to about 1,750 mg, for example, about 700 mg to about 1,400 mg, about 700 mg to about 1,050 mg or about 1,050 mg to about 1,400 mg.

In some embodiments, the antibody (e.g., bispecific antibody) is administered at a dose of about: 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1,000, 1,010, 1,020, 1,030, 1,040, 1,050, 1,060, 1,070, 1,080, 1,090, 1,100, 1,110, 1,120, 1,130, 1,140, 1,150, 1,160, 1,170, 1,180, 1,190, 1,200, 1,210, 1,220, 1,230, 1,240, 1,250, 1,260, 1,270, 1,280, 1,290, 1,300, 1,310, 1,320, 1,330, 1,340, 1,350, 1,360, 1,370, 1,380, 1,390, 1,400, 1,410, 1,420, 1,430, 1,440, 1,450, 1,460, 1,470, 1,480, 1,490, 1,500, 1,510, 1,520, 1,530, 1,540, 1,550, 1,560, 1,570, 1,580, 1,590, 1,600, 1,610, 1,620, 1,630, 1,640, 1,650, 1,660, 1,670, 1,680, 1,690, 1,700, 1,710, 1,720, 1,730, 1,740, 1,750, 1,760, 1,770, 1,780, 1,790, 1,800, 1,810, 1,820, 1,830, 1,840, 1,850, 1,860, 1,870, 1,880, 1,890, 1,900, 1,910, 1,920, 1,930, 1,940, 1,950, 1,960, 1,970, 1,980, 1,990 or 2,000 mg.

In some embodiments, the antibody is administered at a dose of about 700 mg, about 1,050 mg or about 1,400 mg. In some embodiments, the antibody is administered at a dose of about 1,050 mg. In certain embodiments, the antibody is administered at a dose of about 1,400 mg. In particular embodiments, the antibody is administered at a dose of about 700 mg.

In some embodiments, the antibody is administered at a dose of about 350 mg.

In some embodiments, the antibody is administered at a dose of about 750 mg.

In some embodiments, the antibody is administered at a dose of about 800 mg.

In some embodiments, the antibody is administered at a dose of about 850 mg.

In some embodiments, the antibody is administered at a dose of about 900 mg.

In some embodiments, the antibody is administered at a dose of about 950 mg.

In some embodiments, the antibody is administered at a dose of about 1,000 mg.

In some embodiments, the antibody is administered at a dose of about 1,100 mg.

In some embodiments, the antibody is administered at a dose of about 1,150 mg.

In some embodiments, the antibody is administered at a dose of about 1,200 mg.

In some embodiments, the antibody is administered at a dose of about 1,250 mg.

In some embodiments, the antibody is administered at a dose of about 1,300 mg.

In some embodiments, the antibody is administered at a dose of about 1,350 mg.

In certain embodiments, the antibody is administered at a dose of 1,050 mg for body weigh <80 kg and 1,400 mg for body weight≥80 kg.

In particular embodiments, the antibody is administered at a dose of 700 mg for body weigh <80 kg and 1,050 mg for body weight≥80 kg.

In some embodiments, the antibody is administered twice a week.

In certain embodiments, the antibody is administered once a week.

In some embodiments, the antibody is administered once every two weeks.

In certain embodiments, the antibody is administered once every three weeks.

In some embodiments, the antibody is administered once every four weeks.

In certain embodiments, the antibody is administered once a week or once every two weeks. In particular embodiments, the antibody is administered once weekly for the first 4 weeks and then every 2 weeks.

In some embodiments, the antibody is administered on a 28-day cycle.

In some embodiments, the subject has a body weight (BW) of <80 kg, and the antibody (e.g., bispecific antibody such as amivantamab) is administered at a dose of 700 mg once weekly for the first 4 weeks and then every 2 weeks 28-day cycles. In other embodiments, the subject has a body weight of <80 kg, and the antibody (e.g., bispecific antibody such as amivantamab) is administered at a dose of 1,050 mg once weekly for the first 4 weeks and then every 2 weeks 28-day cycles. In some embodiments, the antibody is administered once weekly for the first 4 weeks and then on Days 1 and 15 (28 days cycle). In other embodiments, the subject is administered an IV infusion of amivantamab at a dose of 1,050 or 700 mg if the BW is <80 kg, or 1,400 or 1,050 mg if BW is ≥80 kg, on Days −1, −2, 8, and 22 of Cycle 1 and along with FOLFOX6 chemotherapy (e.g., mFOLFOX6 SoC chemotherapy) on Days 1 and 15 of Cycle 1 and Days 1 and 15 of Cycle 2 (each cycle of 28 days). In other embodiments, the subject is administered an IV infusion of amivantamab along with FOLFIRI chemotherapy on Days −1, −2, and 8 of Cycle 1 and Days 1 and 15 of Cycle 2.

In certain embodiments, the subject has a body weight (BW) of ≥80 kg, and the antibody (e.g., bispecific antibody such as amivantamab) is administered at a dose of 1,050 mg once weekly for the first 4 weeks and then every 2 weeks 28-day cycles. In other embodiments, the subject has a body weight of ≥80 kg, and the antibody (e.g., bispecific antibody such as amivantamab) is administered at a dose of 1.40 mg once weekly for the first 4 weeks and then every 2 weeks 28-day cycles. In other embodiments, the subject administered an IV infusion of amivantamab at a dose of 1,050 or 700 mg if the BW is <80 kg, or 1,400 or 1,050 mg if BW is ≥80 kg, on Days −1, −2, 8, and 22 of Cycle 1 and along with FOLFOX6 chemotherapy (e.g., mFOLFOX6 SoC chemotherapy) on Days 1 and 15 of Cycle 1 and Days 1 and 15 of Cycle 2 (each cycle of 28 days). In other embodiments, the subject is administered an IV infusion of amivantamab along with FOLFIRI chemotherapy on days −1, −2, and 8 of Cycle 1 and Days 1 and 15 of Cycle 2.

Pharmaceutical compositions comprising 1,400 mg, 1,050 mg and 700 mg dose of the anti-EGFR/c-Met antibody can be administered in total volumes of about 28 mL, 21 mL and 14 mL, respectively, with 350 mg/7 mL (50 mg/mL) solution in a single-dose vial.

Additional information regarding amivantamab can be found, for example, in the prescribing information product insert for RYBREVANT® (amivantamab-vmjw) (www.janssenlabels.com/package-insert/product-monograph/prescribing-information/RYBREVANT-pi.pdf), which is incorporated herein by reference.

Additional information regarding the use of amivantamab in patients can be found, for example, in Park K. et al., Amivantamab in EGFR Exon 20 Insertion-Mutated Non-Small-Cell Lung Cancer Progressing on Platinum Chemotherapy: Initial Results From the CHRYSALIS Phase I Study. J Clin Oncol. 2021 Oct. 20; 39(30):3391-3402; Vyse S. Huang P H. Amivantamab for the treatment of EGFR exon 20 insertion mutant non-small cell lung cancer. Expert Rev Anticancer Ther. 2021 Dec. 16; and Cho B C et al., MARIPOSA: phase 3 study of first-line amivantamab+lazertinib versus osimertinib in EGFR-mutant non-small cell lung cancer. Future Oncol. 2021 Dec. 16; which are incorporated herein by reference.

In some embodiments, the antibody is administered as a monotherapy.

### Additional Therapeutic Agents

In some embodiments, the one or more chemotherapeutic agents comprise folinic acid (leucovorin, FOL), fluorouracil (5-FU, F) and oxaliplatin (Eloxatin, OX). FOLFOX, e.g., FOLFOX6, for example, mFOLFOX6, a chemotherapy regimen for treatment of colorectal cancer, is known to those skilled in the art. Additional information regarding FOLFOX can be found, for example, in de Gramont et al., J Clin Oncol. 18(16):2938-47 (2000), Tournigand et al., J Clin Oncol. 22(2):229-37 (2(0)4), Goldberg et al., J Clin Oncol. 22(1):23-30 (2004), Tsai et al., Springerplus. 5(1):1318 (2016), Neugut et al., Clin Colorectal Cancer 18(2):133-40 (2019) and Sobrero et al., Journal of Clinical Oncology 36(15):1478-85 (2018), which are incorporated herein by reference.

In certain embodiments, the one or more chemotherapeutic agents comprise folinic acid (leucovorin, FOL), fluorouracil (5-FU, F) and irinotecan (Camptosar, IRI). FOLFIRI, a chemotherapy regimen for treatment of colorectal cancer, is known to those skilled in the art Additional information regarding FOLFIRI can be found, for example, in Tournigand et al., J Clin Oncol. 22(2):229-37 (2004), Kamnerdsupaphon et al., J Med Assoc Thai. 90(10):2121-7 (2007), Kirstein et al., Oncologist 19(11):1156-68 (2014), Chen et al., Medicine (Baltimore)95(46):e5221 (2016), which are incorporated herein by reference.

In certain embodiments, the method further comprises administering to the subject one or more additional therapeutic agents. Non-limiting examples of the one or more additional therapeutic agents include a T cell expressing chimeric antigen receptor (CAR) (CAR-T cell), a natural killer cell expressing CAR (CAR-NK cell), a macrophage expressing CAR (CAR-M cell), a chemotherapeutic agent, an immune checkpoint inhibitor, a T-cell redirector, radiation therapy, surgery and a standard of care drug. In certain embodiments, the one or more additional therapeutic agents comprises chemotherapy, radiation therapy, surgery, a targeted anti-cancer therapy, a kinase inhibitor, or a combination thereof.

In some embodiments, the one or more additional therapeutic agents are one or more anti-cancer therapies. In some embodiments, the one or more additional therapeutic agents comprise one or more chemotherapeutic agents.

A non-exhaustive list of chemotherapeutic agents considered for use in combination therapies include anastrozole (Arimidex®), bicalutamide (Casodex®), bleomycin sulfate (Blenoxane®), busulfan (Myleran®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin, Rubex®), etoposide (Vepesid®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DT1C-Dome®), fludarabine phosphate (Fludara®), 5-fluorouracil (Adrucil®, Efudex®), flutamide (Eulexin®), teracitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), tamoxifen citrate (Nolvadex®), teniposide (Vumon®), 6-thioguanine, thiotepa, tirapaxamine (Tirazone®), topotecan hydrochloride for injection (Hycamptin®), vinblastine (Velban®), vincristine (Oncovin®), and vinorelbine (Navelbine®).

Example alkylating agents include, without limitation, nitrogen mustards, ethylenimine derivatives, alkyl sulfonates, nitrosoureas and triazenes): uracil mustard (Aminouracil Mustard®, Chlorethaminacil®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmuner®), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), Demethyldopan®, Desmethyldopan®, triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional example alkylating agents include, without limitation, Oxaliplatin (Eloxatin®); Melphalan (also known as L-PAM, L-sarcolysin, and phenylalanine mustard, Alkeran®); Altretamine (also known as hexamethylmelamine (HMM), Hexylen®); Carmustine (BiCNU®); Bendamustine (Treanda®); Busulfan (Busulfex® and Myleran®); Carboplatin (Paraplatin®); Temozolomide (Temodar® and Temodal®); Dactinomycin (also known as actinomycin-D, Cosmegen®); Lomustine (also known as CCNU, CeeNU®); Cisplatin (also known as CDDP, Platnol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexylen®); Ifosfamide (Ofex®); Prednumustine; Procarbazine (Matulane®); Mechlorethamine (also known as nitrogen mustard, mustine and mechloroethamine hydrochloride, Mustargen®); Streptorocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In some embodiments, the one or more additional therapeutic agents comprise a kinase inhibitor. In some embodiments, the kinase inhibitor comprises an inhibitor of EGFR, an inhibitor of c-Met, an inhibitor of HER2, an inhibitor of HER3, an inhibitor of HER4, an inhibitor of VEGFR, an inhibitor of AXL or a combination thereof. In certain embodiments, the kinase inhibitor is an inhibitor of EGFR. In particular embodiments, the kinase inhibitor is an inhibitor of c-Met. In some embodiments, the kinase inhibitor is an inhibitor of HER2. In certain embodiments, the kinase inhibitor is an inhibitor of HER3. In particular embodiments, the kinase inhibitor is an inhibitor of HER4. In some embodiments, the kinase inhibitor is an inhibitor of VEGFR. In certain embodiments, the kinase inhibitor is an inhibitor of or AXL.

In some embodiments, the kinase inhibitor comprises erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib, sunitinib or a combination thereof. In certain embodiments, the kinase inhibitor is erlotinib. In particular embodiments, the kinase inhibitor is gefitinib. In some embodiments, the kinase inhibitor is lapatinib. In certain embodiments, the kinase inhibitor is vandetanib. In some embodiments, the kinase inhibitor is afatinib. In some embodiments, the kinase inhibitor is osimertinib. In certain embodiments, the kinase inhibitor is lazertinib. In particular embodiments, the kinase inhibitor is poziotinib. In some embodiments, the kinase inhibitor is criotinib. In certain embodiments, the kinase inhibitor is cabozantinib. In some embodiments, the kinase inhibitor is capmatinib. In some embodiments, the kinase inhibitor is axitinib. In certain embodiments, the kinase inhibitor is lenvatinib. In some embodiments, the kinase inhibitor is nintedanib. In particular embodiments, the kinase inhibitor is regorafenib. In certain embodiments, the kinase inhibitor is pazopanib. In some embodiments, the kinase inhibitor is sorafenib. In particular embodiments, the kinase inhibitor is sunitinib.

In certain embodiments, the one or more prior anti-cancer therapies comprises carboplatin, paclitaxel, gemcitabine, cisplatin, vinorelbine, docetaxel, palbociclib, crizotinib, PD-(L)1 axis inhibitor, an inhibitor of EGFR, an inhibitor of c-Met, an inhibitor of HER2, an inhibitor of HER3, an inhibitor of HER4, an inhibitor of VEGFR, an inhibitor of AXL, erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib or sunitinib, or any combination thereof.

Anti-cancer therapies that may be administered in combination with the anti-EGFR/c-Met antibody (e.g., bispecific antibody) in the methods of the disclosure include any one or more of the chemotherapeutic drugs or other anti-cancer therapeutics known to those of skill in the art. Chemotherapeutic agents are chemical compounds useful in the treatment of cancer and include growth inhibitory agents or other cytotoxic agents and include alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone: 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL® docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™), vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA®), afatinib (BIBW 2992), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et al, eds.), Chapter 10, McMillan Publishing, are also applicable to the methods of the present invention.

In some embodiments, the anti-EGFR/c-Met antibody (e.g., bispecific antibody) and the one or more additional therapeutic agents (e.g., chemotherapeutic agents) are administered simultaneously. In other embodiments, the antibody and the one or more additional therapeutic agents are administered separately (e.g., sequentially).

For combination therapies, the one or more anti-cancer agents may be administered using recommended doses and dosages of the anti-cancer agent.

### Subjects

The terms “subject” and “patient” can be used interchangeably herein. “Patient in need thereof” or “subject in need thereof” refers to a mammalian subject, preferably human, diagnosed with or suspected of having a disease, to whom will be or has been administered a bi-specific anti-EGFR anti-MET antibody according to a method of the invention. “Patient in need thereof” or “subject in need thereof” includes those subjects already with the undesired physiological change or disease well as those subjects prone to have the physiological change or disease.

In some embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older or 75 years of age or older.

In some embodiments, the subject is a child. In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject has been diagnosed with CRC (e.g., mCRC) for at least about 1 month, e.g., at least about: 2 months, 3 months, 4 months, 5 months, 6 months, 7 months, 8 months, 9 months, 10 months, 11 months, 1 year, 18 months, 2 years, 30 months, 3 years, 4 years, 5 years, 6 years, 7 years, 8 years, 9 years or 10 years. In particular embodiments, the subject is newly diagnosed with CRC (e.g., mCRC). In some embodiments, the CRC is adenocarcinoma.

In certain embodiments, the subject is treatment naïve.

In some embodiments, the subject has received one or more prior anti-cancer therapies. In certain embodiments, the one or more prior anti-cancer therapies comprises one or more chemotherapeutic agents, checkpoint inhibitors, targeted anti-cancer therapies or kinase inhibitors, or any combination thereof. In particular embodiments, the subject is relapsed or resistant to treatment with one or more prior anti-cancer therapies.

In some embodiments, the subject is resistant or has acquired resistance to an EGFR inhibitor. Exemplary EGFR inhibitors for which cancer may acquire resistance are anti-EGFR antibodies cetuximab (ERBITUX®), panitumumab (VECTIBIX®), matuzumab, nimotuzumab, small molecule EGFR inhibitors erlotinib (TARCEVA®), gefitinib (IRESSA®), EKB-569 (pelitinib, irreversible EGFR TKI), pan-ErbB and other receptor tyrosine kinase inhibitors, lapatinib (EGFR and HER2 inhibitor), pelitinib (EGFR and HER2 inhibitor), vandetanib (ZD6474, ZACTIMA™, EGFR, VEGFR2 and RET TKI), PF00299804 (dacomitinib, irreversible pan-ErbB TKI), CI-1033 (irreversible pan-erbB TKI), afatinib (BIBW2992, irreversible pan-ErbB TKI), AV-412 (dual EGFR and ErbB2 inhibitor), EXEL-7647 (EGFR, ErbB2, GEVGR and EphB4 inhibitor), CO-1686 (irreversible mutant-selective EGFR TKI), AZD9291 (irreversible mutant-selective EGFR, TKI), and HKI-272 (neratinib, irreversible EGFR/ErbB2 inhibitor). In some embodiments, the subject is anti-EGFR therapy naïve.

Various qualitative and/or quantitative methods may be used to determine if a subject is resistant, has developed or is susceptible to developing a resistance to treatment with an anti-cancer therapy. Symptoms that may be associated with resistance to an anti-cancer therapy include a decline or plateau of the well-being of the patient, an increase in the size of a tumor, arrested or slowed decline in growth of a tumor, and/or the spread of cancerous cells in the body from one location to other organs, tissues or cells. Re-establishment or worsening of various symptoms associated with cancer may also be an indication that a subject has developed or is susceptible to developing resistance to an anti-cancer therapy, such as anorexia, cognitive dysfunction, depression, dyspnea, fatigue, hormonal disturbances, neutropenia, pain, peripheral neuropathy, and sexual dysfunction. The symptoms associated with cancer may vary according to the type of cancer. For example, symptoms associated with cervical cancer may include abnormal bleeding, unusual heavy vaginal discharge, pelvic pain that is not related to the normal menstrual cycle, bladder pain or pain during urination, and bleeding between regular menstrual periods, after sexual intercourse, douching, or pelvic exam. Symptoms associated with lung cancer may include persistent cough, coughing up blood, shortness of breath, wheezing chest pain, loss of appetite, losing weight without trying and fatigue. Symptoms for liver cancer may include loss of appetite and weight, abdominal pain, especially in the upper right part of abdomen that may extend into the back and shoulder, nausea and vomiting, general weakness and fatigue, an enlarged liver, abdominal swelling (ascites), and a yellow discoloration of the skin and the whites of eyes (jaundice). One skilled in oncology may readily identify symptoms associated with a particular cancer type.

Exemplary PD-(L)1 axis inhibitors are antibodies that bind PD-1 such as nivolumab (OPDIVO®), pembrolimumab (KEYTRUDA®), sintilimab, cemiplimab (LIBTAYO®), tripolibamab, tislelizumab, spartalizumab, camrelizumab, dostralimab, genolimzumab or cetrelimab, or antibodies that bind PD-L1, such as PD-L1 antibodies are envafolimab, atezolizumab (TECENTRIQ®), durvalumab (IMFINZI®) and avelumab (BAVENCIO®).

Marketed antibodies may be purchased via authorized distributor or pharmacy. The amino acid sequences structures of the small molecules can be found from USAN and/or INN submissions by the companies of from CAS registry.

In some embodiments, the subject has EGFR or c-Met expressing cancer.

Exemplary c-Met activating mutations include point mutations, deletion mutations, insertion mutations, inversions or gene amplifications that lead to an increase in at least one biological activity of a c-Met protein, such as elevated tyrosine kinase activity, formation of receptor homodimers and heterodimers, enhanced ligand binding etc. Mutations can be located in any portion of the c-Met gene or regulatory regions associated with the gene, such as mutations in the kinase domain of c-Met. Exemplary c-Met activating mutations are mutations at residue positions N375, V13, V923, R173, V136, L229, S323, R988, S1058/T1010 and E168. Methods for detecting EGFR and c-Met mutations or gene amplifications are well known.

In some embodiments, the subject has been characterized with wild-type KRAS, NRAS and BRAF. In some embodiments, the subject has been characterized with wild-type EGFR.

### Diagnosis

Certain embodiments of the present disclosure concern determining the presence of mutations in a KRAS, NRAS, BRAF, or EGFR gene. Mutation detection methods are known the art, including PCR followed by nucleic acid sequencing, FISH, CGH, or next generation sequencing (NGS). In some embodiments, the mutations are detected by DNA sequencing, such as next generation sequencing (NGS), by using a tumor tissue sample or circulating free DNA from plasma.

In some embodiments, the method comprises:
a) providing a biological sample from the subject;
b) determining presence or absence of a mutation in KRAS, NRAS, BRAF, or EGFR gene in the sample;
c) administering or providing for administration the anti-EGFR/c-Met antibody to the subject determined to have wild type KRAS, NRAS, BRAF, or EGFR gene.

In certain embodiments, the biological sample is a blood sample. In particular embodiments, the biological sample is a tumor tissue biopsy.

In another aspect, the disclosure provides a method of treating mCRC in a subject having wild type KRAS, NRAS, BRAF, or EGFR gene, comprising administering a therapeutically effective amount of an isolated bispecific anti-EGFR/c-Met antibody to the subject, wherein the bispecific anti-EGFR/c-Met antibody comprises a first domain that specifically binds EGFR and a second domain that specifically binds c-Met, wherein the first domain comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In another aspect, the disclosure provides a method of treating mCRC in a subject having wild type KRAS, NRAS, BRAF, or EGFR gene, comprising administering a therapeutically effective amount of an isolated bispecific anti-EGFR/c-Met antibody to the subject, wherein the bispecific anti-EGFR/c-Met antibody comprises a first domain that specifically binds EGFR and a second domain that specifically binds c-Met, wherein the first domain comprises a VH of SEQ ID NO: 13 and a VL of SEQ ID NO: 14; and the second domain comprises the VH of SEQ ID NO: 15 and the VL of SEQ ID NO: 16.

In another aspect, the disclosure provides a method of treating mCRC in a subject having wild type KRAS, NRAS, BRAF, or EGFR gene, comprising administering a therapeutically effective amount of an isolated bispecific anti-EGFR/c-Met antibody to the subject, wherein the bispecific anti-EGFR/c-Met antibody comprises a HC1 of SEQ ID NO: 17, a LC1 of SEQ ID NO: 18, a HC2 of SEQ ID NO: 19 and a LC2 of SEQ ID NO: 20.

In another aspect, the disclosure provides a method of treating mCRC in a subject having wild type KRAS, NRAS, BRAF, or EGFR gene, comprising administering a therapeutically effective amount of an isolated bispecific anti-EGFR/c-Met antibody to the subject, wherein the bispecific anti-EGFR/c-Met antibody is amivantamab.

### EMBODIMENTS

1. A method of treating colorectal cancer n a subject in need thereof, comprising administering a therapeutically effective amount of an anti-epidermal growth factor receptor (EGFR)/hepatocyte growth factor receptor (c-Met) antibody to the subject.
2. The method of embodiment 1, wherein the antibody comprises:
   a) a first domain that specifically binds EGFR, comprising heavy chain complementarity determining region 1 (HCDR1), HCDR2, HCDR3, light chain complementarity determining region 1 (LCDR1), LCDR2 and LCDR3 amino acid sequences of SEQ ID NOs:1, 2, 3, 4, 5 and 6, respectively; and
   b) a second domain that specifically binds c-Met, comprising HCDR1, HCDR2, HCDR3, LCDR1, LCDR2 and LCDR3 amino acid sequences of SEQ ID NOs:7, 8, 9, 10, 11 and 12, respectively.
3. The method of embodiment 2, wherein the first domain comprises a heavy chain variable region (VH) of SEQ ID NO:13 and a light chain variable region (VL) of SEQ ID NO:14, and the second domain comprises a VH of SEQ ID NO:15 and a VL of SEQ ID NO:16.
4. The method of any one of embodiments 1-3, wherein the antibody is of the IgG1 isotype.
5. The method of any one of embodiments 1-4, wherein the antibody comprises a first heavy chain (HC1) of SEQ ID NO:17, a first light chain (LC1) of SEQ ID NO:18, a second heavy chain (HC2) of SEQ ID NO:19 and a second light chain (LC2) of SEQ ID NO:20.
6. The method of any one of embodiments 1-5, wherein the antibody is an isolated bispecific antibody.
7. The method of embodiment 6, wherein the bispecific antibody is amivantamab.
8. The method of any one of embodiments 1-7, wherein the antibody comprises a biantennary glycan structure with a fucose content of about 1% to about 15%.
9. The method of any one of embodiments 1-8, wherein the antibody is administered at a dose of about 700 mg to about 1400 mg.
10. The method of embodiment 9, wherein the antibody is administered at a dose of about 700 mg, about 1,050 mg or about 1,400 mg.
11. The method of embodiment 10, wherein the antibody is administered at a dose of about 1,400 mg.
12. The method of embodiment 10, wherein the antibody is administered at a dose of about 1,050 mg.
13. The method of embodiment 10, wherein the antibody is administered at a dose of about 700 mg.
14. The method of any one of embodiments 1-13, wherein the antibody is administered once a week or once every two weeks.
15. The method of embodiment 14, wherein the antibody is administered once weekly for the first 4 weeks and then every 2 weeks.
16. The method of any one of embodiments 1-15, wherein the antibody is administered on a 28-day cycle.
17. The method of any one of embodiments 1-16, wherein the antibody is administered as a monotherapy.
18. The method of any one of embodiments 1-16, wherein the method further comprises administering one or more chemotherapeutic agents to the subject.
19. The method of embodiment 18, wherein the one or more chemotherapeutic agents comprise FOL FOX, wherein FOLFOX comprises folinic acid, fluorouracil and oxaliplatin.
20. The method of embodiment 18, wherein the one or more chemotherapeutic agents comprise FOLFIRI, wherein FOLFIRI comprises folinic acid, fluorouracil and irinotecan.
21. The method of any one of embodiments 1-20, wherein the colorectal cancer is metastatic colorectal cancer (mCRC).
22. The method of any one of embodiments 1-21, wherein the subject has been characterized with wild-type KRAS, NRAS or BRAF.
23. The method of any one of embodiments 1-22, wherein the subject has been diagnosed with left-sided mCRC.
24. The method of any one of embodiments 1-22, wherein the subject has been diagnosed with right-sided mCRC.
25. The method of any one of embodiments 1-24, wherein the subject is anti-EGFR therapy naïve.
26. The method of any one of embodiments 1-24, wherein the subject has received prior anti-EGFR therapy.
27. The method of any one of embodiments 1-25, wherein the subject is treatment naïve.
28. The method of any one of embodiments 1-26, wherein the subject is relapsed or resistant to treatment with one or more prior anti-cancer therapies.
29. The method of any one of embodiments 1-28, wherein the subject is 18 years of age or older.

### Example 1. Amivantamab in Patients with Advanced or Metastatic Colorectal Cancer

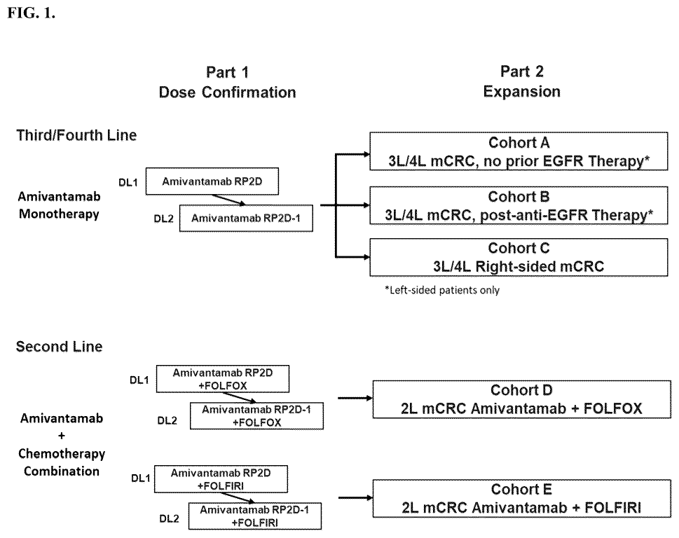

This is an open-label, multicenter Ph1b/2 study of amivantamab as a monotherapy and in combination with chemotherapy in patients with metastatic Colorectal Cancer (mCRC). Part 1 Dose Confirmation will evaluate the safety and confirm the recommended Phase 2 dose (RP2) of amivantamab either as a monotherapy or recommended Phase combination dose (RP2CD) in combination with FOLFOX or FOLFIRI. Part 2 expansion will evaluate the preliminary anti-tumor activity of amivantamab as monotherapy and in combination with FOLFOX or FOLFIRI in the respective populations. Details of the study are described in Table 1 and FIG. 1.

TABLE 1
Amivantamab in Patients with Advanced or Metastatic Colorectal Cancer

| Protocol Number | 61186372GIC2002 | |
|---|---|---|
| Protocol Title | A Phase 1b/2, Open-Label Study of Amivantamab Monotherapy and in | |
| | Combination with Chemotherapy in Patients with Advanced or Metastatic | |
| | Colorectal Cancer. | |
| Synopsis | This is an open-label, multicenter Ph1b/2 study of amivantamab as a | |
| | monotherapy and in combination with chemotherapy in patients with | |
| | metastatic Colorectal Cancer (mCRC), Part 1 Dose Confirmation will | |
| | evaluate the safety and confirm the recommended Phase 2 dose (RP2D) of | |
| | amivantamab either as a monotherapy or recommended Phase 2 combination | |
| | dose (RP2CD) in combination with FOLFOX or FOLFIRI. Part 2 expansion | |
| | will evaluate the preliminary anti-tumor activity of amivantamab as | |
| | monotherapy and in combination with FOLFOX or FOLFIRI in the respective | |
| | populations. | |
| | Amivantamab (also known as Rybrevant ® or JNJ-61186372) is a fully human | |
| | immunoglobulin (Ig) GI-based bispecific antibody (Ab) directed against the | |
| | EGF and MET receptors, with evidence of preclinical activity against NSCLC | |
| | tumors with activating EGFR mutations, the T790M and C797S second-site | |
| | resistance EGFR mutations, overexpressed wild-type EGFR, as well as with | |
| | activation of the MET pathway. | |
| Objectives | Part 1 Dose Confirmation: The primary objective is to characterize the | |
| | safety and tolerability of amivantamab as a monotherapy or in combination | |
| | with chemotherapy in mCRC participants and to characterize the RP2D of | |
| | amivantamab when combined with chemotherapy. | |
| | Part 2 Dose Expansion: The primary objective is to investigate the | |
| | preliminary anti-tumor activity of amivantamab as a monotherapy or in | |
| | combination with chemotherapy in mCRC participants. | |
| | One of the secondary objectives of the study is to assess the additional | |
| | measures of clinical benefit in patients using amivantamab monotherapy or | |
| | amivantamab in combination with chemotherapy. | |
| Overview of | This is an open-label, multicenter, Ph1/2 study of amivantamab as a | |
| Study Design | monotherapy and in combination with chemotherapy in patients with mCRC. | |
| | Study participants must have previously diagnosed with histologically or | |
| | cytologically confirmed unresectable or metastatic adenocarcinoma of the | |
| | colon or rectum. | |
| | Part 1 Dose Confirmation Cohorts | |
| | Amivantamab monotherapy cohorts: Participant must have been | |
| | previously characterized with wild-type KRAS, NRAS, BRAF, and | |
| | either progressed after prior standard of care therapy for metastatic | |
| | disease or be ineligible for all other currently available therapeutic | |
| | options. Participant who has refused all other currently available | |
| | therapeutic options may be eligible (except for participants from | |
| | France) but this must be documented in the study records. | |
| | Part 1 chemotherapy combination cohorts: Participant must have | |
| | been previously characterized with wild-type KRAS, NRAS, BRAF | |
| | and be eligible for treatment with either FOLFOX or FOLFIRI, in | |
| | accordance with standard of care, and be willing to receive additional | |
| | investigational therapy with amivantamab. | |
| | Part 2 Expansion Cohorts | |
| | Amivantamab monotherapy: Participant must have received two or | |
| | three prior lines of systemic therapy for the metastatic setting | |
| | Cohort A: Participant must have been diagnosed with left-sided | |
| | mCRC, which has been previously characterized with wild-type | |
| | KRAS, NRAS, BRAF, and be anti-EGFR therapy naive | |
| | Cohort B: Participant must have been diagnosed with left-sided | |
| | mCRC, which has been previously characterized with wild-type | |
| | KRAS, NRAS, BRAF, and have received prior anti-EGFR therapy | |
| | Cohort C: Participant must have right-sided disease | |
| | Amivantamab + FOLFOX/FOLFIRI: Participant must have received | |
| | one prior line of systemic therapy for the metastatic setting | |
| | Cohort D: Participant must have been diagnosed with left-sided | |
| | mCRC, which has been previously characterized with wild-type | |
| | KRAS, NRAS, BRAF, be anti-EGFR therapy naïve and be a | |
| | candidate to receive FOLFOX | |
| | Cohort E: Participant must have been diagnosed with left-sided | |
| | mCRC, which has been previously characterized with wild-type | |
| | KRAS, NRAS, BRAF, be anti-EGFR therapy naïve and be a | |
| | candidate to receive FOLFIRI | |
| | Study Schema (see FIG. 1) | |
| | Amivantamab Administration: For both Part 1 monotherapy and | |
| | chemotherapy combination cohorts, amivantamab will be administered at | |
| | Dose level 1 (DL1): Starting dose of 1050 mg for body weigh <80 kg | |
| | and 1400 mg for body weight ≥80 kg. | |
| | Dose Level 2 (DL2): The dose of amivantamab may be de-escalated | |
| | to 700 mg for body weight <80 kg and 1050 mg for body weight ≥80 | |
| | kg if DL1 is not deemed tolerable. | |
| | Amivantamab administration will be once weekly for the first 4 weeks and | |
| | then every 2 weeks. | |
| | Anticipated subject enrollment | |
| | Part 1 Dose Confirmation: Approximately 6-12 participants are | |
| | planned to be enrolled each into DL1 and DL2 cohorts for | |
| | amivantamab monotherapy and amivantamab plus chemotherapy | |
| | combination. | |
| | Part 2 Dose expansion: Approximately 40-100 participants are | |
| | expected to be enrolled each into Cohort A, Cohort B, Cohort C, | |
| | Cohort D, and Cohort E | |
| Study Periods | Screening Period: 28 days | |
| | Treatment Period: Begins C1D1 and will continue as 28-day cycles until | |
| | End of Treatment visit (approximately 30 days after discontinuation of | |
| | study treatment) | |
| | Post Treatment Follow Up Follow Period: For participants in Part 2 | |
| | Expansion only | |
| Key Inclusion | 1. | Participant must be at ≥18 years of age. |
| Criteria | 2. | Participant must have been previously diagnosed with histologically or |
| | | cytologically confirmed unresectable or metastatic adenocarcinoma of the |
| | | colon or rectum. |
| | 3. | Participants in each cohort must meet the primary requirements as |
| | | outlined above in section Overview of Study Design, for disease |
| | | characterization and prior treatments. |
| | 4. | For Part 1: Participant must have evaluable disease. |
| | 5. | For Part 2: Participant must have measurable disease according to |
| | | RECIST v1.1. |
| | 6. | ECOG performance status 0 or 1. |
| | 7. | Participant must have adequate organ and bone marrow functions, |
| | | without a history of red blood cell transfusion or platelet transfusion |
| | | within 7 prior to the date of the laboratory test. |
| | 8. | Participant must have tumor lesion amenable for biopsy and agree to |
| | | protocol defined mandatory biopsies. |
| Key Exclusion | 1. | Participant with known HER2 overexpression. |
| Criteria | 2. | Participant with identified mutation in KRAS, NRAS, BRAF, or EGFR |
| | | ectodomain by central ctDNA testing at screening. |
| | 3. | Patients with uncontrolled illness. |
| | 4. | Participant has had prior chemotherapy, targeted cancer therapy, |
| | | immunotherapy, or treatment with an investigational anticancer agent |
| | | within 2 weeks or 4 half-lives whichever is longer or had radiation |
| | | therapy within 4 weeks before the first administration of study treatment. |
| | | For agents with long half-lives, the maximum required time since last |
| | | dose is 28 days. |
| | | Cohort B participants only: At least 3 months must have elapsed since the |
| | | last dose of anti-EGFR therapy prior to enrollment into this study. |
| | 5. | Participants with symptomatic brain metastasis. |
| | 6. | Participant has an active malignancy other than disease under |
| | | investigation. |
| | 7. | Participant has a history of clinically significant cardiovascular disease |
| | 8. | Participant has known allergies, hypersensitivity, or intolerance to |
| | | excipients of amivantamab or has a contraindication to the use of |
| | | FOLFOX (applicable only to participants receiving FOLFOX) or |
| | | FOLFIRI (applicable only to participants receiving FOLFIRI). |
| | 9. | Participant has at screening: |

| | |
|---|---|
| a. | Positive hepatitis B (hepatitis B virus [HBV]) surface |
| | antigen (HBsAg) |
| b. | Positive hepatitis C antibody (anti-HCV [hepatitis C virus]) |
| c. | Other clinically active infectious or non-infectious liver disease |

| | |
|---|---|
| 10. | Participant is known to be positive for human immunodeficiency |
| | virus (HIV) |

| | |
|---|---|
| Key Study | Efficacy: Disease assessments per RECIST 1.1. |
| Measures | Safety: Adverse Events per CTCAE v5.0, clinical safety laboratory |
| | assessments, vital signs. |
| | PK: Serum amivantamab concentrations. |
| | Immunogenicity: Antibodies to amivantamab. |
| PK and | Pharmacokinetic samples will be collected from all subjects enrolled to |
| Immunogenicity | this study (pre- and post-samples for every amivantamab dose). However, |
| | intensive PK collections are required for the first 10 subjects from the |
| | amivantamab monotherapy cohort and for the first 10 subjects from the |
| Assessment | amivantamab + chemotherapy combination cohort. |
| | Samples required (intensive PK) |
| | C1: D1, D2, D4, D8, D11, D15, D22 |
| | C2: D1, D2, D4, D8, D11, D15 |
| | C3: D1, D15 |
| | C4: D1, D2, D4, D8, D11, D15 |
| | C7, C10, C13: D1, D15 |
| | Then every 12 cycles after C13 (i.e. C25, C37 etc.) |
| | End of Treatment |
| | Post Treatment Follow Up: First visit only |

### Example 2. Amivantamab Monotherapy in Addition to Standard-of-Care Chemotherapy in Participants with Advanced or Metastatic Colorectal Cancer

This is an open-label, multicenter Phase 1b/2 study of amivantamab as a monotherapy and in combination with chemotherapy in patients with metastatic Colorectal Cancer (mCRC) as described in Table 2.

TABLE 2
Amivantamab Monotherapy and in Addition to Standard-of-Care Chemotherapy
in Participants with Advanced or Metastatic Colorectal Cancer

| Study ID | CR109215 | |
|---|---|---|
| | 2021-006629-23 | |
| | 61186372GIC2002 | |
| Title | A Phase 1b/2, Open-Label Study of Amivantamab Monotherapy and in | |
| | Addition to Standard-of-Care Chemotherapy in Participants with Advanced or | |
| | Metastatic Colorectal Cancer. | |
| Brief | The purpose of this study is to assess the anti-tumor activity of amivantamab | |
| Summary | as a monotherapy (Cohorts A, B, and C), to characterize the safety of | |
| | amivantamab when added to standard-of-care (SoC) chemotherapy in | |
| | participants with metastatic colorectal cancer (mCRC) (Ph2 cohorts), and to | |
| | assess the recommended phase 2 combination dose (RP2CD) of amivantamab | |
| | when added to SoC chemotherapy (Ph1b cohorts). | |
| Detailed | Colorectal cancer (CRC) is a major global health concern and the third most | |
| Description | common cancer worldwide. Amivantamab (also known as RYBREVANT or | |
| | JNJ-61186372) is a fully human immunoglobulin (Ig) G1-based bispecific | |
| | antibody (Ab) directed against epidermal growth factor (EGF) and | |
| | mesenchymal epithelial transition (MET) receptors, with evidence of | |
| | preclinical activity against non-small cell lung cancer (NSCLC) tumors with | |
| | activating EGF receptor (EGFR) mutations, the T790M and C797S second- | |
| | site resistance EGFR mutations, overexpressed wild-type EGFR, as well as | |
| | with activation of the MET pathway. Amivantamab has demonstrated activity | |
| | in both EGFR- and MET-driven NSCLC, with preclinical evidence | |
| | demonstrating its ability to recruit immune effector cells. While two anti- | |
| | EGFR antibodies are incorporated as part of the SoC for CRC patients, MET | |
| | is highly expressed or amplified in subsets of CRC and additionally plays a | |
| | role in mediating resistance to anti-EGFR treatments. The study consists of up | |
| | to 28 days screening period, treatment period will begin on Cycle 1 Day 1 | |
| | (C1D1) (for Cohorts A, B, and C) or C1D -2 (for Ph1b-D, Ph1b-E, Cohorts D | |
| | and E) with the administration of the study treatment and continue as 28-day | |
| | cycles until the end of the treatment visit, up to 30 days after discontinuation | |
| | of study treatment. The safety of amivantamab as a monotherapy or in | |
| | addition to SoC chemotherapy will be assessed by physical examinations, | |
| | Eastern Cooperative Oncology Group (ECOG) criteria for performance status | |
| | (PS) laboratory tests, vital signs, monitoring of adverse events, and | |
| | concomitant medication usage. The total duration of this study will be up to 4 | |
| | years 1 month. | |
| Arms | Cohorts A, B, and C: Amivantamab Monotherapy (Experimental) | |
| | Participants in Cohort A (no prior anti-epidermal growth | |
| | factor receptor [EGFR] therapy), Cohort B (post anti-EGFR | |
| | therapy), and Cohort C (with or without anti-EGFR therapy), | |
| | will be administered intravenous (IV) infusion of | |
| | amivantamab 1050 milligrams (mg) if body weight (BW) is | |
| | less than (<) 80 kilograms (kg) or 1400 mg if BW is greater | |
| | than or equal to (>=) 80 kg, as monotherapy on Days 1 and | |
| | 15 of Cycle 2 (28-days cycle). | |
| | Cohorts Ph1b-D and D: Amivantamab + 5-Fluorouracil, Leucovorin, and | |
| | Oxaliplatin (mFOLFOX6 (Active Comparator) | |
| | Participants who are anti-EGFR treatment naïve, have not | |
| | received oxaliplatin-based chemotherapy in the metastatic | |
| | setting, will be administered IV infusion of amivantamab | |
| | 1050 or 700 mg (dose level 0 [DL0]) if BW is <80 kg, or | |
| | 1400 or 1050 mg (dose de-escalation [DL-1]) if BW is >=80 | |
| | kg, on Days −1, −2, 8, and 22 of Cycle 1 and along with | |
| | mFOLFOX6 SoC chemotherapy on Days 1 and 15 of Cycle 1 | |
| | and Days 1 and 15 of Cycle 2 (each cycle of 28 days) in | |
| | Phase 1b dose confirmation Cohort (Cohort Ph1b-D). | |
| | Participant in Phase 2 Cohort (Cohort D) will receive | |
| | recommended Phase 2 combination dose (RP2CD) of | |
| | amivantamab along with mFOLFOX6 SoC chemotherapy | |
| | determined in Cohort Ph1b-D. | |
| | Cohorts Ph1b-E and E: Amivantamab + 5-Fluorouracil, Leucovorin, and | |
| | Irinotecan (FOLFIRI) (Active Comparator) | |
| | Participants who are anti-EGFR treatment naïve, have not | |
| | received irinotecan-based chemotherapy in the metastatic | |
| | setting, will be administered IV infusion of amivantamab | |
| | along with FOLFIRI SoC chemotherapy on Days −1, −2, and | |
| | 8 of Cycle 1 and Days 1 and 15 of Cycle 2 in Ph1b-E. For | |
| | Cohort E, RP2CD determined in Ph1b-E will be | |
| | administered. | |
| Inclusion | Sex: all | |
| Criteria | Not Gender Based | |
| | Age Limits: Minimum Age: 18 years; Maximum Age: N/A | |
| | Healthy volunteers are not accepted | |
| | Participant must have been previously diagnosed with histologically or | |
| | cytologically confirmed unresectable or metastatic adenocarcinoma of the | |
| | colon or rectum | |
| | For Phase 1 dose confirmation cohorts (Cohorts Ph1b-D and Ph1b-E): | |
| | Participant must have evaluable disease. For Phase 2 dose expansion | |
| | cohorts (Cohorts D and E): Participant must have measurable disease | |
| | according to Response Criteria in Solid Tumors (RECIST) Version 1.1. If | |
| | only one measurable lesion exists, it may be used for the screening biopsy | |
| | as long as baseline tumor assessment scans are performed greater than or | |
| | equal to (>=) 7 days after the biopsy | |
| | Participant must have Eastern Cooperative Oncology Group (ECOG) | |
| | performance status (PS) 0 or 1 | |
| | Participant must have a tumor lesion amenable for biopsy and agree to | |
| | mandatory protocol-defined screening biopsy | |
| | A female participant of childbearing potential must have a negative serum | |
| | pregnancy test at screening and within 72 hours of the first dose of study | |
| | treatment and must agree to further serum or urine pregnancy tests during | |
| | the study | |
| Exclusion | 11. | Participant with known Erb-B2 receptor tyrosine kinase 2 (ERBB2)/ |
| Criteria | | human epidermal growth factor receptor 2 (HER-2) amplification based |
| | | on the local testing results |
| | 12. | Participant with identified mutation in Kirsten rat sarcoma viral oncogene |
| | | (KRAS), neuroblastoma RAS viral oncogene homolog (NRAS), v-raf |
| | | murine sarcoma viral oncogene homolog B (BRAF), or epidermal growth |
| | | factor receptor (EGFR) ectodomain, or ERBB2/HER2 amplification by |
| | | central circulating tumor deoxyribonucleic acid (ctDNA) testing at |
| | | screening |
| | 13. | Participant with symptomatic brain metastasis |
| | 14. | History or known presence of leptomeningeal disease |
| | 15. | Any condition for which, in the opinion of the investigator, participation |
| | | would not be in the best interest of the participant (for example, |
| | | compromise the well-being) or that could prevent, limit, or confound the |
| | | protocol-specified assessments |

| | |
|---|---|
| Interventions | Amivantamab (RYBREVANT ®; JNJ-61186372) (Type: Drug) |
| | Associated Arms: |
| | Cohorts A, B, and C: Amivantamab Monotherapy |
| | Cohorts Ph1b-D and D: Amivantamab + 5-Fluorouracil, Lencovorin, and |
| | Oxaliplatin (mFOLFOX6) |
| | Cohorts Ph1b-E and E: Amivantamab + 5-Fluorouracil, Leucovorin, and |
| | Irinotecan (FOLFIRI) |
| | Amivantamab will be administered as intravenous infusion |
| | Fluorouracil (Type: Biological/Vaccine) |
| | Associated Arms: |
| | Cohorts Ph1b-D and D: Amivantamab + 5-Fluorouracil, Leucovorin, and |
| | Oxaliplatin (mFOLFOX6) |
| | Cohorts Ph1b-E and E: Amivantamab + 5-Fluorouracil, Leucovorin, and |
| | Irinotecan (FOLFIRI) |
| | Fluorouracil will be administered as intravenous infusion |
| | Leucovorin (Type: Biological/Vaccine) |
| | Associated Arms: |
| | Cohorts Ph1b-D and D: Amivantamab + 5-Fluorouracil, Leucovorin, and |
| | Oxaliplatin (mFOLFOX6) |
| | Cohorts Ph1b-E and E: Amivantamab + 5-Fluorouracil, Leucovorin, and |
| | Irinotecan (FOLFIRI) |
| | Leucovorin will be administered as intravenous infusion |
| | Oxaliplatin (Type: Biological/Vaccine) |
| | Associated Arms: |
| | Cohorts Ph1b-D and D: Amivantamab + 5-Fluorouracil, Leucovorin, and |
| | Oxaliplatin (mFOLFOX6) |
| | Oxaliplatin will be administered as intravenous infusion |
| | Irinotecan (Type: Biological/Vaccine) |
| | Associated Arms: |
| | Cohorts Ph1b-E and E: Amivantamab + 5-Fluorouracil, Leucovorin, and |
| | Irinotecan (FOLFIRI) |
| | Irinotecan will be administered as intravenous infusion |
| Primary | Cohorts A, B, and C: Objective Response Rate (ORR) |
| Outcome | Time Frame: Up to 4 years 1 month |
| Measures | ORR is defined as the percentage of participants who achieve either a |
| | partial response (PR) or complete response (CR), as defined by |
| | investigator assessment using Response Criteria in Solid Tumors |
| | (RECIST) version 1.1. |
| | Cohorts Ph1b-D and Ph1b-E: Number of Participants with Dose-limiting |
| | Toxicity (DLT) |
| | Time Frame: Up to 4 years 1 month |
| | Number of participants with DLT will be assessed. The DLTs are specific |
| | adverse events and are defined as any of the following: high grade non- |
| | hematologic toxicity, or hematologic toxicity. |
| | Cohorts Ph1b-D and Ph1b-E: Number of Participants with DLT by |
| | Severity |
| | Time Frame: Up to 4 years 1 month |
| | Number of participants with DLT by severity will be assessed. The DLTs |
| | are specific adverse events and are defined as any of the following: high |
| | grade non-hematologic toxicity, or hematologic toxicity. Toxicities will |
| | be graded for severity according to the National Cancer Institute- |
| | Common Terminology Criteria for Adverse Events (NCI-CTCAE) |
| | version 5.0, graded as Grade 1: mild, Grade 2: moderate, Grade 3: severe, |
| | Grade 4: life-threatening, and Grade 5: death related to adverse event. |
| | Cohorts D and E: Number of Participants Adverse Events (AE) |
| | Time Frame: Up to 4 years 1 month |
| | An AE is any untoward medical occurrence in a participant participating |
| | in a clinical study that does not necessarily have a causal relationship |
| | with the pharmaceutical/ biological agent under study, Severity of AEs |
| | will be graded according to the NCI-CTCAE version 5.0. Severity scale |
| | ranges from Grade 1 (Mild) to Grade 5 (Death). Grade 1: mild, Grade 2: |
| | moderate, Grade 3: severe, Grade 4: life-threatening and Grade 5: death |
| | related to adverse event. |
| | Cohorts D and E: Number of Participants with Laboratory Values |
| | Abnormalities |
| | Time Frame: Up to 4 years 1 month |
| | Number of participants with laboratory values abnormalities, which |
| | includes serum chemistry, hematology, coagulation, and urinalysis, will |
| | be reported. |
| | Cohorts D and E: Number of Participants with Vital Signs Abnormalities |
| | Time Frame: Up to 4 years 1 month |
| | Number of participants with vital signs including temperature, heart rate, |
| | respiratory rate, and blood pressure (systolic and diastolic) and oxygen |
| | saturation, abnormalities will be reported. |
| Secondary | Cohorts A, B, C, Ph1b-D, and Ph1b-E: Number of Participants with AEs |
| Outcome | Time Frame: Up to 4 years 1 month |
| Measures | An AE is any untoward medical occurrence in a participant participating |
| | in a clinical study that does not necessarily have a causal relationship |
| | with the pharmaceutical/biological agent under study. Severity of AEs |
| | will be graded according to the NCI-CTCAE version 5.0. Severity scale |
| | ranges from Grade 1 (Mild) to Grade 5 (Death), Grade 1: mild, Grade 2: |
| | moderate, Grade 3: severe, Grade 4: life-threatening and Grade 5: death |
| | related to adverse event. |
| | Cohorts A, B, C, Ph1b-D, and Ph1b-E: Number of Participants with |
| | Laboratory Values Abnormalities |
| | Time Frame: Up to 4 years 1 month |
| | Number of participants with laboratory values abnormalities, which |
| | includes serum chemistry, hematology, coagulation, and urinalysis, will |
| | be reported. |
| | Cohorts A, B, C, Ph1b-D, and Ph1b-E: Number of Participants with |
| | Vital Signs Abnormalities |
| | Time Frame: Up to 4 years 1 month |
| | Number of participants with vital signs including temperature, heart rate, |
| | respiratory rate, and blood pressure (systolic and diastolic) and oxygen |
| | saturation, abnormalities will be reported. |
| | Cohorts Ph1b-D, Ph1h-E, D and E: ORR |
| | Time Frame: Up to 4 years 1 month |
| | ORR is defined as the percentage of participants who achieve either a PR |
| | or CR, as defined by investigator assessment using RECIST version 1.1. |
| | Cohorts Ph1b-D, Ph1b-E, D, and E: Duration of Response (DoR) |
| | Time Frame: Up to 4 years 1 month |
| | DoR is defined as the time from the date of first documented response |
| | (PR or CR) until the date of documented progression or death, whichever |
| | comes first, for participants who have PR or CR, as defined by |
| | investigator assessment using RECIST version 1.1. |
| | Cohorts Ph1b-D, Ph1b-E, D, and E: Clinical Benefit Rate (CBR) |
| | Time Frame: Up to 4 years 1 month |
| | CBR is defined as the percentage of participants achieving complete or |
| | partial response, as well as durable stable disease (defined as a duration of |
| | at least 11 weeks) as defined by RECIST version 1.1. |
| | Cohorts D and E: Progression Free Survival (PES) |
| | Time Frame: Up to 4 years 1 month |
| | PFS is defined as the time from the first administration of study treatment |
| | until the date of objective disease progression or death, whichever comes |
| | first, based on investigator assessment using RECIST version 1.1. |

1. A method of treating colorectal cancer in a subject in need thereof, the method comprising administering a therapeutically effective amount of an anti-epidermal growth factor receptor (EGFR)/hepatocyte growth factor receptor (c-Met) antibody to the subject to thereby treat the colorectal cancer, wherein the antibody comprises:
a first domain that specifically binds EGFR comprising a heavy chain complementarity determining region 1 (HCDR1) comprising the amino acid sequence of SEQ ID NO: 1, a HCDR2 comprising the amino acid sequence of SEQ ID NO: 2, a HCDR3 comprising the amino acid sequence of SEQ ID NO: 3, a light chain complementarity determining region 1 (LCDR1) comprising the amino acid sequence of SEQ ID NO: 4, a LCDR2 comprising the amino acid sequence of SEQ ID NO: 5, and a LCDR3 comprising the amino acid sequence of SEQ ID NO: 6; and
a second domain that specifically binds c-Met, comprising a HCDR1 comprising the amino acid sequence of SEQ ID NO: 7, a HCDR2 comprising the amino acid sequence of SEQ ID NO: 8, a HCDR3 comprising the amino acid sequence of SEQ ID NO: 9, a LCDR1 comprising the amino acid sequence of SEQ ID NO: 10, a LCDR2 comprising the amino acid sequence of SEQ ID NO: 11, and a LCDR3 comprising the amino acid sequence of SEQ ID NO: 12.

2. (canceled)

3. The method of claim 1, wherein the first domain comprises a heavy chain variable region (VH) comprising the amino acid sequence of SEQ ID NO: 13 and a light chain variable region (VL) comprising the amino acid sequence of SEQ ID NO: 14, and the second domain comprises a VH comprising the amino acid sequence of SEQ ID NO: 15, and a VL comprising the amino acid sequence of SEQ ID NO: 16.

4. The method of claim 1, wherein the antibody is of the IgG1 isotype.

5. The method of claim 1, wherein the antibody comprises a first heavy chain (HC1) comprising the amino acid sequence of SEQ ID NO: 17, a first light chain (LC1) comprising the amino acid sequence of SEQ ID NO: 18, a second heavy chain (HC2) comprising the amino acid sequence of SEQ ID NO: 19, and a second light chain (LC2) comprising the amino acid sequence of SEQ ID NO: 20.

6. The method of claim 1, wherein the antibody is an isolated bispecific antibody.

7. The method of claim 6, wherein the bispecific antibody is amivantamab.

8. The method of claim 1, wherein the antibody comprises a biantennary glycan structure with a fucose content of about 1% to about 15%, or less than 20%.

9. The method of claim 1, wherein the antibody is administered at a dose of about 700 mg to about 1,400 mg.

10. The method of claim 9, wherein the antibody is administered at a dose of about 700 mg, about 1,050 mg or about 1,400 mg.

11. The method of claim 10, wherein the antibody is administered at a dose of about 1,400 mg.

12. The method of claim 10, wherein the antibody is administered at a dose of about 1,050 mg.

13. The method of claim 10, wherein the antibody is administered at a dose of about 700 mg.

14. The method of claim 1, wherein the antibody is administered once a week or once every two weeks.

15. The method of claim 14, wherein the antibody is administered once weekly for the first 4 weeks and then every 2 weeks.

16. The method of claim 1, wherein the antibody is administered on a 28-day cycle.

17. The method of claim 1, wherein the antibody is administered as a monotherapy.

18. The method of claim 1, wherein the method further comprises administering one or more chemotherapeutic agents to the subject.

19. The method of claim 18, wherein the one or more chemotherapeutic agents comprise FOLFOX, wherein FOLFOX comprises folinic acid, fluorouracil and oxaliplatin.

20. The method of claim 18, wherein the one or more chemotherapeutic agents comprise FOLFIRI, wherein FOLFIRI comprises folinic acid, fluorouracil and irinotecan.

21. The method of claim 1, wherein the colorectal cancer is metastatic colorectal cancer (mCRC).

22-24. (canceled)

25. The method of claim 1, wherein the subject is anti-EGFR therapy naïve.

26. The method of claim 1, wherein the subject has received prior anti-EGFR therapy.

27. The method of claim 1, wherein the subject is treatment naïve.

28. The method of claim 1, wherein the subject is relapsed or resistant to treatment with one or more prior anti-cancer therapies.

29. (canceled)
